(19) United States
(12) Patent Application Publication
(10) Pub. No.: US 2008/0027405 A1
(43) Pub. Date: Jan. 31, 2008

(54) Absorbent articles and printed wipes

(76) Inventors: Rosa Alejandra Hernandez, Cincinnati, OH (US); Ilonka Bibiana Laviz, Mason, OH (US); Irene Spalazzi, Mason, OH (US); Ronald Bosman Visscher, Cincinnati, OH (US)

Correspondence Address: THE PROCTER & GAMBLE COMPANY;INTELLECTUAL PROPERTY DIVISION - WEST BLDG., WINTON HILL BUSINESS CENTER - BOX 412, 6250 CENTER HILL AVENUE, CINCINNATI, OH, 45224, US

(21) Appl. No.: 11/495,299

(22) Filed: Jul. 28, 2006

(51) Int. Cl. A61F 13/15

(52) U.S. Cl. 604/385.06; 604/385.02

(57) ABSTRACT

An absorbent article combination comprising an absorbent article and a printed wipe. The printed wipe can be individually packaged and attached to an individually packaged absorbent article.

### FIELD OF THE INVENTION

The present invention relates generally to the field of absorbent articles and wipes.

### BACKGROUND OF THE INVENTION

Absorbent articles, such as sanitary napkins, liners, and tampons, are generally used by women to absorb menstrual or other body exudates. Diapers and incontinence articles are used by babies or other incontinent individuals to absorb and contain body exudates, such as urine and/or feces. Often, the use and changing of these articles is messy and unhygienic. The hands or body of the person changing the article can become contaminated due to a failure of the article to contain the exudates or due to exudates that reside on the user's body or garments after removal of the article. In addition, it can be desirable to clean portions of the user's body or garments, which can also lead to contamination of the hands of the person changing the article. Thus, it would be beneficial to have a means for cleaning soiled skin and/or garments while using or changing an absorbent article.

One means for cleaning soiled surfaces is a wipe. Such wipes can include, for example, wet wipes or dry wipes. Wet wipes used for cleansing are often made of a nonwoven material and comprise a liquid cleansing solution. Some wipes contain a fragrance and/or a lotion. Typically, such wet wipes are generally uniform in color and rectangular in shape. Multiple wipes are often packaged together in a common package, but wipes can be individually packaged.

Because catamenial absorbent articles and adult incontinence pads are often changed in a restroom, the use and changing of such articles can be awkward, especially when the consumer is in a public restroom. The mechanics of changing the article while cleaning the body and attempting to prevent contamination of the user's hands by the body exudates and/or the surfaces of the public restroom can be challenging and stressful. Even when the consumer has a wipe available, the consumer may not be able to quickly appreciate how the wipe is to be used or may not feel fresh and clean even after using the wipe. Thus, using the absorbent article and/or a wipe may not be enjoyable for a user. Further, many consumers do not currently use wet wipes when using or changing an absorbent article and may not use the wipe even if provided with the article if the consumer cannot quickly appreciate the benefits of the wipe and how it is to be used.

As such, it would be desirable to provide a combination absorbent article and wipe product that communicates product benefits to a user. It would also be desirable to provide a product that educates the user as to the use of the wipe/article combination. Furthermore, it would be desirable to provide a wipe that makes using the wipe while changing or during the use of an absorbent article more simple and enjoyable.

### SUMMARY OF THE INVENTION

An absorbent article combination comprising an absorbent article and a pouch joined to the absorbent article, the pouch having a wipe disposed therein, wherein the wipe includes a substrate that is printed, is provided. The wipe can be printed with at least one image that provides a signal of at least one predetermined functional characteristic of the wipe, such as, for example, freshness, cleanliness, lotion, scent, odor control, an emotional benefit, and/or combinations thereof. The wipe can also or alternatively be printed to provide a signal to educate a consumer about at least one predetermined fact, to coordinate with printing or coloration of the absorbent article, and/or to communicate how and/or when to use a wipe when changing or using an absorbent article. The wipe can be a nonwoven material and/or a wet wipe.

### BRIEF DESCRIPTION OF THE DRAWINGS

FIG. 1 is a cross-sectional side view of one embodiment of the present invention.

FIG. 2 is a perspective view of an alternative embodiment of the present invention.

FIG. 3 is a cross-sectional view of one embodiment of the present invention.

FIG. 4 is an isometric view of one embodiment of the present invention.

FIG. 5 is an isometric view of one embodiment of the present invention.

FIG. 6 is an isometric view of one embodiment of the present invention.

### DETAILED DESCRIPTION OF THE INVENTION

The present invention relates to printed wipes that can be used in conjunction with an absorbent article. It is desirable to provide a wipe that allows a consumer to quickly and reliably identify the benefits of a wipe when changing or using the associated absorbent article. When appropriate signals are used, the benefits of the wipe and how to use the wipe can be relatively well-understood by the user. Therefore, the present invention provides wipes, such as, e.g., wet wipes, that can be printed to communicate product benefits, to educate the user, to provide an emotional benefit, and/or to communicate how and/or when to use a wipe when changing or using an absorbent article. For example, the wipes can be printed to communicate product benefits, such as, e.g., the presence of lotion and/or scent on the wipe and/or a feeling of freshness and/or cleanliness to the user. The wipes can be printed to educate the user about the wipe or the associated absorbent article; or about the menstrual cycle, urinary-incontinence, or baby care, depending on the nature of the associated absorbent article; or about other information relevant to the user. In certain embodiments, the wipe can be printed to provide an emotional benefit, such as, e.g., a message conveying a relaxed feeling, a happy or pleasant feeling, and/or an encouraged feeling. The wipes can also, or alternatively, be printed to indicate which side of the wipe should be utilized, such as, e.g., to maximize cleaning efficacy and minimize contamination of the user's hand. In certain embodiments, the printed wipe can be individually packaged and the individually packaged wipe can be joined to an individually packaged absorbent article.

The combination of a wipe with an absorbent article can provide one or more of several advantages. For example, the wipe can provide for cleansing, which can provide the wearer with increased comfort, physically and/or emotionally. The use of the wipe can also provide a reduction in the odor associated with menstruation or other bodily exudates. In addition, the use of the wipe can provide emotional benefits by providing a feeling of freshness and/or cleanliness.

As used herein, the term “absorbent article” refers to devices that absorb and/or contain a substance, such as, e.g., body exudates. A typical absorbent article can be placed against or in proximity to the body of the wearer to absorb and contain various body exudates. “Individually packaged,” as used herein, means that only one item is packaged in a particular package.

“Absorbent article combination” as used herein refers to an absorbent article with a wipe joined thereto.

As used herein the term “joined” refers to the condition where a first member or component is attached, affixed, or otherwise physically connected to a second member or component either directly or indirectly. An example of an indirectly joined member or component might be where the first member or component is affixed or connected to an intermediate member or component that in turn is affixed or connected to the second member or component. The joined relationship between the first member or component and the second member or component can be fixedly or releasably.

As used herein the term “fixedly joined” refers to a connection that cannot be unattached without at least partially destroying one of the attached components. As used herein the term “releasably joined” refers to a connection that is meant to be easily released.

As used herein, the term “wipe” refers to a piece of material that can be used to clean a user's skin or a surface. The wipe made of any suitable material, such as, e.g., nonwoven material, paper material, woven material, knitted material, tufted material, stitch-bonded material incorporating binding yarns or filaments, or material felted by wet-milling. Nonwoven material as used herein is “a manufactured sheet, web or batt of directionally or randomly orientated fibers, bonded by friction, and/or cohesion and/or adhesion, excluding paper and products which are woven, knitted, tufted, stitch-bonded incorporating binding yarns or filaments, or felted by wet-milling.” “ISO 9092:1988, Textiles-Nonwovens-Definition” International Organization for Standardization, 1988. As such, the wipe can be a “nonwoven wipe,” that is, a wipe comprising a nonwoven substrate. The wipe can also be a “paper wipe,” that is, a wipe comprising a paper substrate, such as, e.g., a wet-laid substrate like toilet tissue or facial tissue.

The term “wet wipe” as used herein refers to a wipe that includes a substrate that is moistened with any fluid prior to packaging.

The term “image” when used with reference to printing includes, for example, any type of mark, figure, picture, identification code, symbol, icon, pattern, or text, such as, e.g., a word, number, nomenclature, sentence, or instruction. An image can also be a line, line segment, curved line, band, arrow, area of coloration, or any other printed indicia having a purpose of providing a signal or guide to the user.

As used herein, the term “design” when used with reference to printing refers to one or more images. A design can include one image or a combination of images or image types, such as, for example, multiples of the same image type or a combination of image types. A combination of image types could include, e.g., at least one figure and at least one word. The design can also be a pattern, such as, for example, a shaded, striped, or stippled pattern.

As used herein, the term “functional characteristic” when used with respect to a wipe refers to a benefit that the wipe provides to a user. A functional characteristic can be an actual characteristic, such as, e.g., a lotion or scent that is applied to a wipe, or a perceived characteristic, such as, e.g., an emotional benefit and/or a feeling the user experiences when viewing or using the wipe.

As used herein, the term “emotional benefit” refers to a functional characteristic that promotes or enhances an intended feeling in the user, such as, for example, a feeling of well-being and/or psychological comfort. An emotional benefit can include, for example, a relaxed feeling, a happy or pleasant feeling, an encouraged feeling, a feeling of joy, wonder, happiness, amusement, courage, intimacy, relaxation, security, appreciation, love, familiarity, alertness, strength, hope, calmness, contentment, confidence, delight, gladness, hope, nostalgia, peace, affection, anticipation, enthusiasm, combinations thereof, or any other feeling associated with a user's well-being.

By “visually perceptible” is meant that a human viewer can visually discern the image and/or design with the unaided eye (excepting standard corrective lenses adapted to compensate for near-sightedness, farsightedness, or astigmatism, or other corrected vision) in lighting at least equal to the illumination of a standard 100 watt incandescent light bulb at a distance of 1 meter.

By “only visually perceptible” is meant that the image and/or design cannot be readily perceived by touch, sound, or smell. Therefore, channels, embossments, tufts, folds, pleats, and other tactilely-perceptible elements of an absorbent article or wipe would not considered to be only visually perceptible if they can be perceived by, e.g., touch.

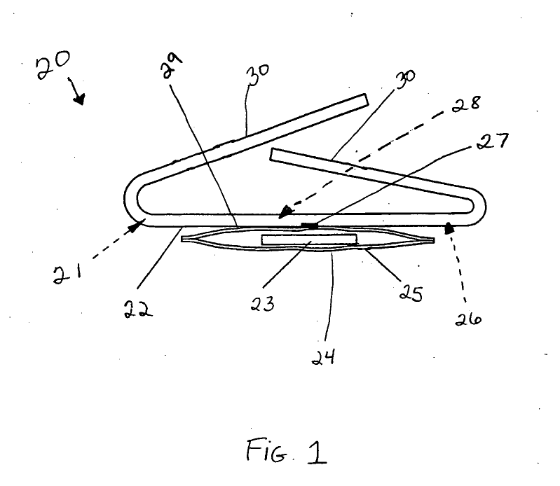

FIG. 1 shows a cross-sectional side view showing an absorbent product combination 20 that comprises a sanitary napkin 21 and an individually packaged wipe 23 that is in a pouch 25. In this embodiment, the pouch 25 is joined to the sanitary napkin 21 by a joining means 27. The pouch 25 can be joined to the sanitary napkin 21 either releasably or fixedly. Any suitable means for joining the pouch 25 to the sanitary napkin 21 can be used. One example of a suitable joining means 27 is an adhesive, such as, e.g., a pressure sensitive adhesive. In certain embodiments, the sanitary napkin 21 and pouch 25 are releasably joined using a glue attachment, such as, for example, hot melt glue. The sanitary napkin 21 can have a releasable wrapper 22 that is either releasably joined to or not joined to the sanitary napkin 21. The releasable wrapper 22 can either partially or completely surround the sanitary napkin 21. In certain embodiments, the pouch 25 is releasably attached to the releasable wrapper 22 of the sanitary napkin 21. The pouch 25 can also, or alternatively, be fixedly joined to the sanitary napkin 21.

The sanitary napkin 21 shown in FIG. 1 has a body-facing side 28 and an opposed garment-facing side 26. The pouch 25 has an outwardly facing side 24 and an opposed inwardly facing side 29. The inwardly facing side 29 of the pouch 25 can be joined to the garment facing side 26 of the sanitary napkin 21. FIG. 1 shows the pouch 25 joined to the sanitary napkin 21 in the middle of the garment-facing side 26. The pouch 25 can be joined to the sanitary napkin 21 at any suitable location on the sanitary napkin 21, the releasable wrapper 22, or a package enclosing the sanitary napkin 21. In certain embodiments, the pouch 25 is joined to the sanitary napkin 21 at one end 30 of the napkin 21. Alternatively, the pouch 25 could be located on the body-facing side 28 of the sanitary napkin 21. These locations can permit the pouch 25 containing the wipe 23 to be removed and used before or without opening the sanitary napkin 21.

In certain embodiments, the sanitary napkin 21 can comprise a pair of flaps that each extend from a longitudinal side margin. Suitable sanitary napkins are described in, for example, U.S. Pat. Nos. 5,346,486; 5,009,653; 4,950,264; 4,917,697; 4,687,478; 4,589,876; and 4,285,343. In this embodiment, the flaps of the sanitary napkin 21 are folded over the topsheet in a topsheet facing relationship, and the pouch 25 containing the wipe 23 is releasably affixed to the flaps such that the topsheet facing relationship is maintained. Such an arrangement is described in, for example, U.S. Pat. No. 6,911,022.

Any pouch 25 suitable for holding a wipe 23 can be used. In certain embodiments, the pouch 25 can be separate from the absorbent article prior to attachment. For example, a pouch separate from an absorbent article may not comprise a component of the absorbent article. In other embodiments, the releasable wrapper 22 can comprise a flap (or pouch) to contain a wipe 23. A suitable flap (or pouch) is described in, for example, U.S. Pat. Nos. 4,556,146 and 5,569,230. The absorbent article can comprise a releasable paper wrapper 22 that surrounds the absorbent article to form an individual package and the pouch 25 comprises a portion of the releasable paper wrapper 22, such as, for example, the pouches described in, e.g., U.S. Pat. No. 5,569,230.

With respect to the sanitary napkin 21 shown in FIG. 1, the sanitary napkin 21 can be individually packaged, such as, for example, in a flexible poly wrapper. In certain embodiments, the releasable paper wrapper 22 of the sanitary napkin 21 can surround the sanitary napkin 21 to form a package. The individually wrapped sanitary napkins can be packaged in a retail package, such as, for example, a box or bag, such as a poly bag.

Although FIG. 1 shows a sanitary napkin 21 as the absorbent article, any suitable absorbent article can be used, such as, e.g., pantiliners, incontinence articles, interlabial pads, or baby diapers. Suitable pantiliners are disclosed in U.S. Pat. No. 4,738,676. Other absorbent articles are described in, for example, U.S. Pat. Nos. 6,059,764; 5,951,536; 5,824,004; 5,702,382; 5,683,375; 5,658,269; 5,439,458; 5,304,161; and 5,300,054.

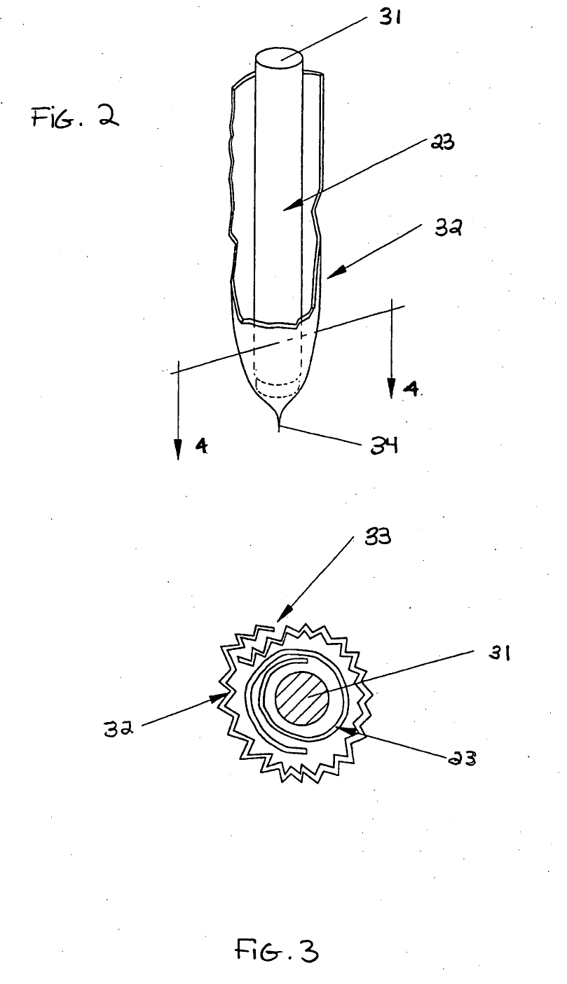

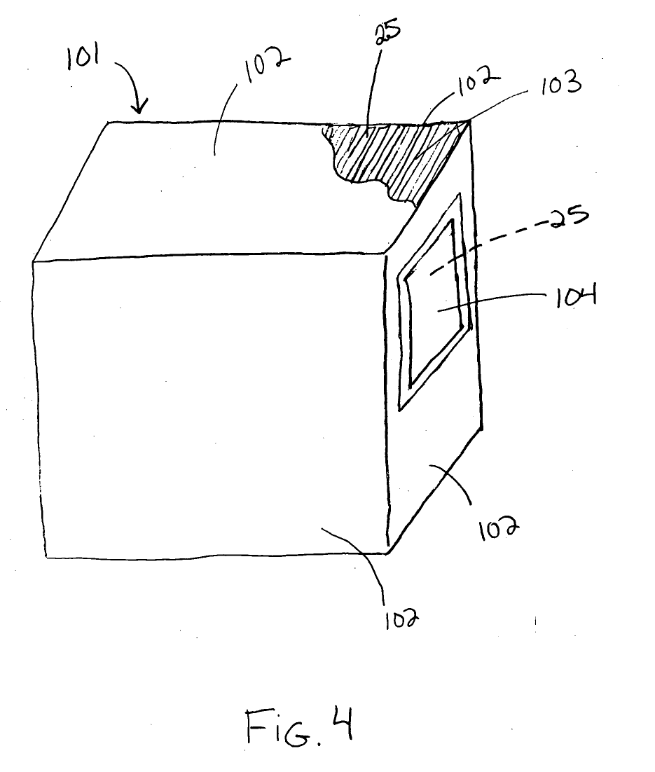

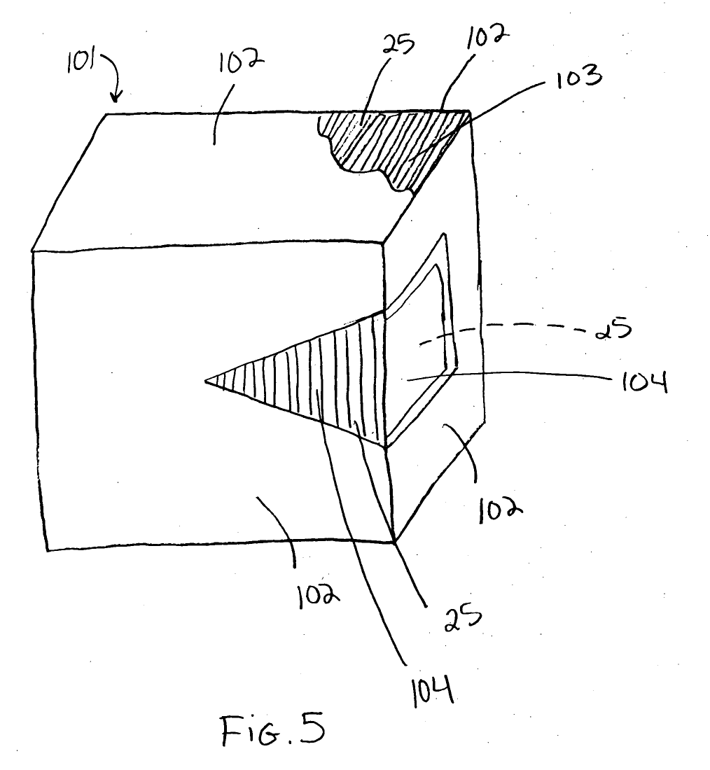

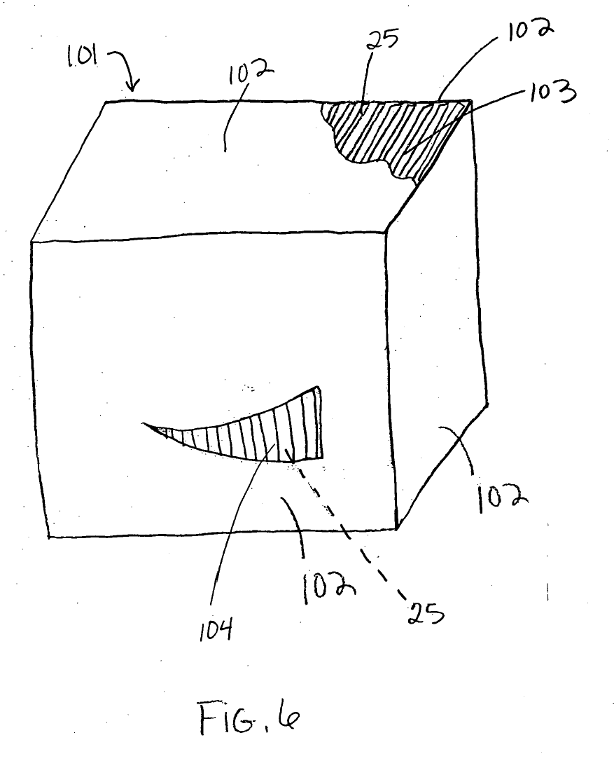

FIGS. 2 and 3 show an example of an embodiment for packaging a wipe 23 with a tampon 31. The tampon 31 can be any known form, such as, for example, tampons disclosed in U.S. Pat. No. 4,475,911. The wipe 23 can be wrapped around the tampon 31. The wrapped tampon 31 can be wrapped with a moisture and vapor impervious overwrap 32. The side 33 and ends (one of which is shown as 34) of the overwrap 32 are sealed. The tampon package can be provided with a releasable seal at the side 33, and/or at least one of the ends 34 for use in opening the package. Any suitable configuration and/or means for joining the tampon 31 and the wipe 23 can be used. For example, in certain embodiments, the personal wipe 23 can be releasably attached to the overwrap 32 of the tampon 31.

In certain embodiments, the wipe 23 can be printed to communicate product benefits, such as, e.g., a predetermined functional characteristic, to the user. The wipe 23 can be printed with a design that communicates product benefits such as, for example, freshness, cleanliness, the presence of lotion, scent, and/or odor control. Any suitable design that communicates relevant benefits can be used, including images, such as, for example, plants such as flowers and/or flower petals, animals such as cats, insects such as butterflies, shapes, characters such as cartoon characters, scenes, seasonal things or goods, and/or other nature images, such as ocean waves, water drops, sun rays or images of the moon or stars. The image itself can communicate product benefits and/or the images can be arranged in a pattern to form a design that communicates product benefits. For example, the wipe 23 can be printed with one or more flowers to communicate freshness, a scent, and/or odor control.

In certain embodiments, the wipe 23 can be printed to provide emotional benefit(s) when the wipe 23 is used or viewed by a user. Such emotional benefits include, for example, a relaxed feeling, a happy or pleasant feeling, or an encouraged feeling. Any suitable image or design can be used to provide such emotional benefits, such as, e.g., pleasant images or other images or designs described herein. In certain embodiments, the image can be text that can be combined with another image type or types if desired. For example, the wipe 23 could be printed with an encouraging message that could be combined with an image such as, for example, a flower.

In certain embodiments, the wipe 23 can be printed with at least one image to educate the consumer. The wipe 23 can be printed with a design that communicates a story or a fact to the consumer. For example, the design could include images such as, e.g., text, icons, or symbols that provide the consumer with a useful fact of relevance to the user, such as, e.g., a fact about menstrual cramps, incontinence, or baby care. The wipe 23 could also or alternatively be printed with design that educates the user about the benefits of using the wipe 23, such as images such as, for example, words or symbols that convey freshness or cleanliness and/or staying fresh longer, or symbols to communicate the presence of an ingredient, such as, e.g., blue dots to communicate the presence of aloe and/or diamonds to communicate the presence of odor absorbing particles. In certain embodiments, the wipe 23 can be printed with at least one image to educate the consumer about the product, such as, for example, with information about the wipe 23 and/or absorbent article and/or about the scent, cleaning ability, and/or wetness of the wipe 23.

In certain embodiments, the wipe 23 can be printed to communicate to the user which side of the wipe 23 should be used. When a wipe 23 has enhanced benefits on one side, such as, e.g., texture and/or lotion, the consumer can have difficulty observing which side of the wipe 23 should be utilized during the cleaning process. Thus, in certain embodiments, one side of the wipe 23 is printed. The other side of the wipe 23 can be free of printing or can be printed with a different design. Because one side of the printed wipe 23 is distinct from the other side of the wipe 23, the user can orient the wipe 23 so as to use the proper surface of the wipe 23 to clean the contaminated body areas. In this embodiment, the wipe 23 can have enhanced benefits on one side, such as, e.g., texture and/or lotion, and the wipe 23 can be printed to communicate to the user which side of the wipe 23 has the enhanced benefits. In certain embodiments, one side of the wipe 23 can be dry, such as, for example, to prevent and/or reduce wetness on a user's hands during use, and the other side of the wipe 23 can be wet, such as, e.g., for improved cleaning. The wipe 23 can have a barrier between the two sides. Either side of the wipe 23 can be printed on, as long as the communication is clear to the user as to which side of the wipe 23 has the enhanced benefits. Thus, in certain embodiments, the wipe 23 can be printed on the side of the wipe 23 with the enhanced benefits. In certain embodiments, the wipe 23 has a first printing on the side of the wipe 23 with the enhanced benefits and a second printing on the reverse side of the wipe 23. For example, the wipe 23 could be printed with images such as dots or flowers on the side of the wipe 23 with enhanced benefits and with lines in a pattern on the reverse side or vice versa. In certain embodiments, usage instructions are printed on the side of the wipe 23 with enhanced benefits. In certain embodiments, the wipe 23 is printed such that the user perceives that the indicated portion of the wipe 23 can be one of greater moisturizing, fluid removal, improved cleaning, disinfection, improved softness, or other signals that facilitate an emotional response or proper use of the wipe 23.

In certain embodiments, an array of wipes is provided. At least some of the wipes 23 in the array can be printed with different images to communicate to the user, such as, e.g., a feeling of excitement and/or interest to learn what design is printed on the next wipe 23 in the array. Such printing can encourage the user to use the wipe 23 while changing or using the absorbent article and/or provide a more enjoyable and/or exciting cleaning experience for the user. The wipe 23 can also, or alternatively, be printed to communicate a “fortune” or inspiring statement to a user. In certain embodiments, a first wipe 23 is printed with a first design comprising at least one image, a second wipe 23 is printed with a second design comprising at least one image, and so on throughout the series. The wipes can be printed with more than one image or design such that each wipe 23 in the array comprises a design that is different from other wipes in the array. In certain embodiments, different wipes in the array have a common theme, such as, for example, a theme of flowers and/or a coloration or printing that is similar or complementary in shade and/or color. Any suitable number of wipes can be in the array, such as, e.g., about 5, about 10, about 15, about 20, about 25, or any other suitable number. In certain embodiments, the array of wipes comprises the number of wipes a woman is expected to use during a single menstrual cycle. In certain embodiments, the array of wipes comprises a number of wipes that is the same as the number of absorbent articles packaged with the wipes or co-marketed therewith. In certain embodiments, each wipe 23 in the array is individually packaged and attached to an individually packaged absorbent article. In certain embodiments, more than one wipe 23 can be attached to one absorbent article and/or one wipe 23 can be attached to more than one absorbent article.

In certain embodiments, the wipe 23 is printed to communicate to the user that the wipe 23 should be used with coordinated absorbent articles. Such printing assists the user in appreciating that a wipe 23 should be used when using or changing an absorbent article and also to quickly select the proper wipe 23 for use based on the printing and/or coloration of the wipe 23 or pouch 25 if the wipe 23 is separately packaged from the absorbent article. The wipe 23, absorbent article, and exterior packaging can each be printed in a coordinating fashion to communicate that the wipe 23 and absorbent article should be used together. For example, the wipe 23, absorbent article, and exterior packaging could each be printed with the same design and/or have printing or coloration in the same shade. In such an arrangement, the printing can be disposed on, for example, the wipe 23, the pouch 25, the absorbent article, the releasable wrapper 22, a package containing the absorbent article, and/or an exterior packaging such as, for example, a bag or a box in which a plurality of absorbent article combinations are packaged. In certain embodiments, the wipe 23, pouch 25, absorbent article, and/or package can be printed with information about the corresponding absorbent article and/or wipe 23.

The wipe 23, pouch 25, or wipe exterior package can also or alternatively be printed with a design that communicates that wipe 23 coordinates with the absorbent article to which it is attached. For example, a wipe 23 packaged with a sanitary napkin 21 designed for overnight use could be printed with a moon and/or stars. In another embodiment, the wipe 23 can be printed to coordinate and/or correspond to printing and/or coloration on the absorbent article or absorbent article packaging to which the wipe 23 is joined. For example, a wipe 23, packaged with a liner that is printed with purple flowers could also be printed with purple flowers. Alternatively, a wipe 23 packaged with a liner that is printed with purple flowers could be printed with other images or text in a shade similar to the purple used on the liner.

In certain embodiments, the wipe 23 can be individually packaged and can be joined to the absorbent article. One individually packaged wipe 23 can be joined to one individually packaged absorbent article, more than one individually packaged wipe 23 can be joined to one individually packaged absorbent article, one individually packaged wipe 23 can be joined to more than one absorbent article that can be individually packaged, more than one wipe packaged together can be joined to one or more individually packaged absorbent articles, one or more individually packaged wipes can be joined to one or more absorbent articles packaged together, or any suitable combination. In certain embodiments, the wipe can be individually packaged, but is not joined to the absorbent article. For example, the wipes can be packaged in individual pouches and a plurality of individual pouches containing wipes can be packaged with absorbent articles in the same exterior packaging or the plurality of individual pouches can be packaged in a different exterior package from that of the absorbent article. The wipes and the absorbent articles can be co-marketed, such as, e.g., designed to be sold and/or used together, such as, for example, for the same purpose. Alternatively, a plurality of wipes can be packaged together in a common package and then packaged with absorbent articles in the same exterior packaging or the plurality of wipes can be packaged together in a common package that is different from that of the absorbent article. In certain embodiments, a plurality of individually packaged wipes are packaged with a plurality of individually packaged absorbent articles wherein the individual package of one or both of the absorbent article or the wipe is provided with an attachment means that allows attachment of the wipe 23 to the absorbent article, such as described in, for example, WO 2005/115295.

In certain embodiments, a plurality of wipes is packaged together. The wipes can be packaged in individual pouches and then packaged in any suitable packaging. Alternatively, the wipes can be packaged together, such as, for example, in a stack, and then packaged in packaging, such as, for example, a hard-sided container or tub; or a soft-sided package such as a pouch or bag. In certain embodiments, the wipes are packaged together in a stack and at least one wipe 23 in the stack is printed to communicate a reminder to buy more wipes. For example, a wipe 23 or wipes can be printed with an image or text signaling that a refill should be purchased and/or the number of wipes remaining in the package. The printed wipe 23 or wipes can be visually perceptible by the user after about 50%, about 75%, about 80%, about 85%, about 90%, or about 95% of the wipes in the package have been used.

In certain embodiments, wipes are printed so that the consumer can distinguish between wipes that are otherwise substantially identical in appearance. For example, each wipe 23 can differ in odor control, sensate delivery, lotion delivery, skin care agent concentration, wetness, or any other attribute beneficial in the context of a wipe 23. Printing the wipes with different designs can allow the user to more readily identify wipes of differing characteristics, and therefore more easily choose the appropriate product for the user's particular needs. In addition, such designs can provide the user with the ability to take visual notice before or while using a wipe 23 to identify the wipe 23 for future use and/or purchase.

The wipe 23 can be printed with any suitable image or design. Any suitable colors and/or letter fonts can be used. The colors of printed regions can be uniform, or they can be varying shades or hues of one color, or they can be different colors. In certain embodiments, the wipe 23 is printed in a color or shade coordinating with the attached or coordinating absorbent article.

The wipe 23 can be printed using any printing technology suitable to add color to the wipe 23, such as, for example, gravure printing, offset printing, inkjet printing, and combinations thereof, and in any suitable manner, such as, for example, discrete or continuous printing. The printing can add color in specific areas or in random areas. In certain embodiments, color is applied only by printing, such as by ink-jet printing, onto the wipe 23, and does not include a previously colored or dyed component, such as a uniformly-colored nonwoven. In certain embodiments, the wipe 23 is a wet wipe and the wipe 23 is printed while dry, i.e., prior to the addition of cleansing solution.

The wipe 23 can be printed in any suitable location. For example, the wipe 23 can be printed in different places or can have continuous printing in a center area (e.g., in the center, cross direction) or discrete printing (e.g., always in the center cross and machine direction). The printing can be in the central portion of the wipe 23, off center, diagonally across the wipe 23, or on the perimeter of the wipe 23. In certain embodiments, the wipe 23 is printed in an area having enhanced benefits. The wipe 23 can be printed on both sides or on one side. In certain embodiments, a series of wipes is provided wherein at least some of the wipes are printed in a different location on the wipe 23 from the other wipes in the series.

Any suitable ink can be used to print on a wipe 23. Suitable inks include compositions comprising a colorant such as, e.g., a dye or pigment, that is suspended, dispersed or dissolved in a solvent, for example, water. The ink can be aqueous or non-aqueous. In certain embodiments, the ink comprises a pigment, a cross-linking polymer, a cross-linking agent, and, optionally, a solvent. The ink can also comprise modified soybean oils, waxes, or mixtures thereof.

The wipes can be made such that, rather than printing with ink, the wipe 23 material is modified so as to have a different reflective index, or to be transparent, in the region intended to be printed. Likewise, instead of ink, color can be added by adding colored material in appropriate places. The colored material can include films, nonwovens, or adhesives, including glue and hot melt adhesives or combinations of these or other materials. Such color can be added in or on any component of the wipe 23, as long as it is visible to the user when the wipe 23 is being used.

In certain embodiments, the wipe 23 can be embossed. At least one or all of the embossed depressions can have therein a substance such as ink to provide visible color. The visible color can be printed in registration with the embossments, or printed at the same time as the embossments are made by means known in the art, such as the method described in WO 04/057110, or U.S. Pat. No. 6,780,270. The color can be due to printing inks, dyes, paint, or colored adhesive. The color can also be on a separate layer, such that upon embossing, color shows through the embossed portion, thereby giving the appearance of having been printed in registration with the embossment. In certain embodiments, the wipe 23 can comprise a substrate that includes one or more colored fibers. The colored fibers can be incorporated into the substrate such that they are visible to a user, such as, e.g., in the form of one or more images.

The wipe 23 can be a nonwoven wipe comprising a substrate comprising a nonwoven web of natural fibers, synthetic fibers, or mixtures of natural and synthetic fibers wherein the substrate is not considered paper. Suitable natural fibers include, e.g., cellulosic fibers, such as wood pulp fibers, cotton, and rayon. Suitable synthetic fibers include fibers commonly used in textiles, such as, for example, polyolefins, such as polyester and polypropylene fibers. In certain embodiments, the wipe 23 is a nonwoven wipe comprising a substrate that comprises viscose rayon and/or wood pulp. Alternatively, the wipe 23 can be a paper wipe, such as, e.g., a wipe 23 comprising a substrate that is made of a wet-laid material in a manner similar to toilet tissue or facial tissue. In certain embodiments, the wipe can comprise silk.

The wipe 23 can be “flushable”, that is, it is of a size of is capable of disintegrating to pieces sufficiently small such that when flushed in a toilet and when being transported in the sewer system, it does not plug any element of a sewer system. Any wrapping material associated with the wipe 23, such as, for example, the pouch 25 material, can also be flushable. Wrapping materials that meet such criteria can include, for example, water soluble materials, such as, e.g., silicone-treated polyvinyl alcohol films, films coated with a polyvinyl alcohol, and tissue coated or impregnated with polyvinyl alcohol. Suitable materials are disclosed in, e.g., U.S. Pat. No. 4,755,421.

The wipe 23 can be any size suitable for cleansing the skin or providing other benefits when using or changing an absorbent article. In certain embodiments, the wipe 23 can be rectangular. The wipe 23 can be greater than or equal to about 4 square inches (about 25 square centimeters) in size, greater than or equal to about 9 square inches (about 50 square centimeters) in size, less than or equal to about 225 square inches (about 1,450 square centimeters) in size, between about 16 square inches (about 100 square centimeters) and about 50 square inches (about 320 square centimeters), or about 35 square inches (about 225 square centimeters) in size.

The wipe 23 can be a wet wipe that comprises a liquid-based cleansing solution. The wipe 23 can also be scented. In certain embodiments, the wipe 23 can comprise a nonwoven fabric impregnated with the cleansing solution. The cleansing solution can include, for example, surfactants, alcohols, fragrance compositions, antimicrobial agents, pH buffers, and combinations thereof. The wipe 23 can also contain substances such as silicones that can inhibit body exudates from adhering to the user's body. The cleaning solution can also contain skin conditioning substances similar to those used in hand lotions, such as, e.g., aloe and Vitamin E; other substances for applying to the skin, such as, e.g., witch hazel; or any other substances known in the art for inclusion in wet wipes.

The wipe 23 can comprise a lotion, such as, for example, an aqueous or non-aqueous lotion. In certain embodiments, the lotion can increase the cleaning capabilities of the wipe 23 by aiding removal of soiling from the skin. The lotion can also provide skin benefits by reducing friction between the skin and the wipe 23 and/or by containing materials that can soothe the skin, such as, for example, aloe or chamomile. Examples for suitable lotions are disclosed in, for example, EP 808151; EP 763,341, and WO 00/57843.

One or more fragrance compositions can be included in the cleansing solution in order to produce a scented wipe 23. Alternatively, the one or more fragrance compositions can be included in the wipe 23 separately from any cleansing solution such as, for example, by applying the fragrance composition to the wipe 23 prior to or after any cleansing solution can be added. The wipe 23 can also include a separate component such as, for example, a carrier material comprising a fragrance composition. Additionally, the one or more fragrance compositions can be disposed together or separately on at least a portion of the wipe 23.

Other embodiments can include dry wipes or wipes containing non-aqueous cleaning solutions such as, e.g., mineral oils, and the emollient described in U.S. Pat. No. 4,481,243.

In certain embodiments, the wipe 23 can be packaged in a pouch 25. Any suitable pouch 25 for holding a wipe 23 can be used. For example, the pouch 25 can be made of paper and/or other suitable materials, such as, e.g., polyethylene. When the wipe 23 is a wet wipe, any pouch 25 suitable for maintaining the wetness of the wipe 23 can be used. For example, the pouch 25 can comprise at least one barrier material suitable for preventing evaporation of moisture from the wipe 23 can be used. Suitable barrier materials include, for example, laminates with a layer of ethylene vinyl alcohol (EVOH), propylene vinyl alcohol (PVOH), or aluminum sandwiched between polymeric film layers, such as LDPE (low density polyethylene); metallized, such as aluminized plastic films; metal foils; oriented polyethylene terephtalate (PET); PETG (glycol-modified PET); oriented polyamide; aromatic polyamide; or polymeric films like polyethylene films. In certain embodiments, the pouch 25 comprises paper and/or foil. The pouch 25 can also, or alternatively, comprise an inner layer comprising polyethylene (PE), an outer layer comprising a copolymer of polyethylene (PE) and polypropylene (PP) and a subjacent intermediate layer comprising polypropylene (PP).

In certain embodiments, one or more sides of the pouch 25 can be sealed to prevent substantial evaporation of the liquid solution from the time the product is manufactured to the time it is used by the consumer. Any number of sealing mechanisms can be used including, for example, heat and/or pressure seals, ultrasonic seals, glue seals, zippered track sealing systems such as Dow Company's “ZIP LOCKS” or combinations thereof. In certain embodiments, the pouch 25 can be hermetically sealed. In addition, the pouch 25 can be-lined with foil, or other suitable material to further reduce evaporation.

The pouch 25 can include a means for unsealing (or opening) the pouch 25 in order to access a wipe 23 contained therein. For example, the pouch 25 can have a scored or die cut line of weakness to allow the pouch 25 to be opened easily with a tab, easy peel label or any other opening mechanism; a “Dry-edge” sticker mechanism, such as, e.g., a sticker with a dry edge for grasping; releasable adhesives; releasable heat and/or pressure seals; perforations; rupture seals; tear seals; or the pouch 25 can be made of a relatively easy to tear material such as, for example, an easy to tear film.

Any suitable joining means can be used to attach the pouch 25 and absorbent article of the absorbent article combination. In certain embodiments, the joining means on the outside of the package of the wipe 23 can be selected and applied such that a sufficient degree of joining between the package of the absorbent article and the package of the wipe 23 is generated. Any suitable adhesive can be used to join the absorbent article and the wipe 23, such as, for example, a pressure-sensitive adhesive, hot melt adhesive, cold melt adhesive, or solvent-based adhesives. The adhesive can be applied to the absorbent article and/or wipe 23 as a continuously coated area or as a pattern, such as, e.g., dots, stripes, spirals, or beads. In certain embodiments, the absorbent article and the wipe 23 can be joined by one or more mechanical fasteners. Suitable mechanical fasteners include, for example, hook and loop fasteners.

The pouch 25 including a wipe 23 contained therein, the wipe 23, and/or the absorbent article can be packaged in an exterior package. One or more items can be packaged in the exterior package. Any suitable package can be used, such as, e.g., a bag and/or a box.

In certain embodiments, wipes 23 are contained in an exterior package. FIG. 8 shows a package 101 having one or more walls 102 that define an interior storage space 103 for storing one or more wipes 23. One or more wipes 23 can be packaged in a pouch 25, such as, for example, individually packaged in a pouch 25. In certain embodiments, more than one wipe 23 can be packaged in each pouch 25. The package 101 can be made of one or more inflexible materials, such as, for example, cardboard, paperboard, cartonboard, chipboard, plywood, SBS, metal, plastic, paper, card stock, fabric, ceramic, polymer, natural or synthetic fibers, webs, mesh, screen, wood, composite, mixtures or combinations thereof, or any other suitable material. Alternatively, or in addition, the package 101 can be made of a flexible material, such as, e.g., blown or cast film in a blend of low density polyethylene and linear low density polyethylene, metallocenes, ethylene vinyl acetate, surlyn, polyethylene terephthalate, biaxally oriented polypropylene, nylon, combinations thereof, or any other suitable material. In certain embodiments, the package 101 can be constructed of paper laminate film comprising low density polyethylene, aluminum foil, polyethylene adhesive, and/or paper. The package 101 can be any suitable shape, such as, for example, a square, a rectangle, and/or a polyhedral shape. The wipes 23 and/or pouches 25 can all be identical to one another or one or more wipes 23 and/or one or more pouches 25 can be different from each other. In certain embodiments, the package 101 can comprise one or more first wipes that can be packaged in a first pouch 25 and one or more second wipes that can be packaged in a second pouch 25.

The wipes 23 and/or pouches 25 can be arranged to form a stack, such as, e.g., a pile, within the interior space 103 of the package 101. The wipes 23 and/or pouches 25 can be stacked in any direction. For example, the wipes 23 and/or pouches 25 can be stacked vertically, horizontally, or at any angle inside the interior storage space 103 of the package 101.

The package 101 can include a means for accessing the interior storage space 103 for easy access to the wipes 23 and/or pouches 25. The mechanism or means for accessing the interior storage space 103 can be any suitable mechanism, such as, for example, a lid, a tab, a line of perforations, adhesive openings, or combinations thereof.

The package 101 can include at least one region of transparency 104, such as shown in FIG. 8. As used herein, “transparency” means having the property of transmitting light without substantial scattering so that items lying beyond can be visible to a person. The region of transparency 104 can comprise a transparent covering, such as, for example, a transparent film. In addition, or alternatively, the region of transparency 104 can comprise an opening in the package 101 that does not include a covering. In certain embodiments, the region of transparency 104 can comprise a transparent covering that can be tinted. The transparent covering can be tinted in any suitable color or combination of colors. The transparent covering can also, or alternatively, be printed and/or embossed.

The region of transparency 104 can be disposed on at least one side, such as, e.g., the front, the back, and/or either side. In certain embodiments, the region of transparency 104 can be disposed on more than one side. For example, the region of transparency 104 can form a continuous window on two adjacent walls, such as, e.g., shown in FIG. 9. The region of transparency 104 can be at any location on the side and can comprise any appropriate amount of the package side, such as, for example, from about 5% to about 95% of the side, such as, for example, about 5% to about 75%, about 5% to about 50%, about 5% to about 25%, or any other suitable amount of the side. In certain embodiments, the region of transparency 104 can be located in the central region of a side, such as, for example, wherein the region of transparency 104 is not located at the edge of the side.

The region of transparency 104 can be any suitable shape. Suitable shapes includes, e.g., a polygon, such as, e.g., a three-sided polygon, a triangle, a square, a rectangle, and/or a quadrilateral shape. For example, the region of transparency 104 can be substantially in the shape of a triangle and/or a three-sided polygon. In certain embodiments, the region of transparency 104 can be a tapered shape, such as, for example, the shape shown in FIGS. 9-10. In certain embodiments, the region of transparency can be an irregular shape. The irregular shape can be substantially in the shape of one or more shapes, such as, for example, a polygon, such as, e.g., a three-sided polygon, a triangle, a square, a rectangle, and/or a quadrilateral shape.

In certain embodiments, the wipe 23 can be positioned adjacent the region of transparency 104 such that at least a portion of the wipe 23 and, optionally, a portion of any images contained thereon, can be visible through the region of transparency 103. One or more wipes 23 can be packaged in a pouch 25 and the pouch 25 can be positioned adjacent the region of transparency 104 such that at least a portion of the pouch 25 and, optionally, a portion of any images contained thereon, can be visible through the region of transparency 104. In certain embodiments, the package 101 can have one or more regions of transparency 104 located on one or more package walls 102, wherein at least one wipe 23 that can be packaged in a pouch 25 is positioned adjacent at least one of the one or more regions of transparency 104 such that at least a portion of the wipe 23 and/or pouch 25 and/or at least a portion of one or more images included thereon are visible to a human through at least one of the one or more regions of transparency 104. In certain embodiments, the package 101 can contain an array of wipes 23 and/or pouches 25, wherein the array comprises at least a first wipe and/or pouch and a second wipe and/or pouch, wherein the first wipe and second wipe are different, can be positioned adjacent at least one of the one or more regions of transparency 104 such that at least a portion of the first and second wipe and/or pouch are visible to a human through at least one of the one or more regions of transparency 104.

In certain embodiments, the package 101 can include one or more images 105. For example, the package 101 can include a depiction of a wipe 106 having a water drop 107 disposed thereon and can also, or alternatively, include images 105, such as, for example, a depiction of one or more water droplets 108, that can relate to the one or more images 109 on the one or more wipes 23 that can be packaged in a pouch 25 that are viewable by a user through one or more regions of transparency 104.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm”.

All documents cited in the Detailed Description of the Invention are, in relevant part, incorporated herein by reference; the citation of any document is not to be construed as an admission that it is prior art with respect to the present invention. To the extent that any meaning or definition of a term in this written document conflicts with any meaning or definition of the term in a document incorporated by reference, the meaning or definition assigned to the term in this written document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. An absorbent article combination comprising:
a. an absorbent article; and
b. a pouch joined to the absorbent article, the pouch having a wipe disposed therein, wherein the wipe comprises a substrate that is printed.

2. The absorbent article combination of claim 1, wherein the substrate is printed with at least one image that provides a signal of at least one predetermined functional characteristic of the wipe.

3. The absorbent article combination of claim 2, wherein the functional characteristic is selected from the group consisting of: freshness, cleanliness, lotion, scent, odor control, and combinations thereof.

4. The absorbent article combination of claim 2, wherein the functional characteristic is an emotional benefit.

5. The absorbent article combination of claim 1, wherein the substrate is printed with at least one image that provides a signal to educate a consumer about at least one predetermined fact.

6. The absorbent article combination of claim 5, wherein the predetermined fact includes a fact about the absorbent article or the wipe.

7. The absorbent article combination of claim 1, wherein the absorbent article comprises at least one printing or coloration, and wherein the substrate is printed to coordinate with the printing or coloration of the absorbent article.

8. The absorbent article combination of claim 7, wherein the substrate and the absorbent article are printed with the same design or have printing or coloration in the same shade.

9. The absorbent article combination of claim 8, wherein the combination further comprises an exterior package wherein the exterior package is printed with the same design as the substrate and the absorbent article.

10. A package comprising:
a. an absorbent article; and
b. a wipe comprising a substrate that is printed.

11. The package of claim 10, wherein the wipe is individually packaged.

12. The package of claim 10, wherein the substrate is printed with at least one image selected from the group consisting of: a signal of at least one predetermined functional characteristic of the wipe; a signal to educate a consumer about at least one predetermined fact; an image coordinating with printing or coloration of the absorbent article.

13. An array of nonwoven wipes, wherein the array comprises a plurality of wipes, each wipe comprising a substrate, the array comprising at least a first wipe and a second wipe, wherein the first wipe is printed with a first design comprising at least one image and the second wipe is printed with a second design comprising at least one image, wherein the first design and the second design are visibly different.

14. The array of claim 13, wherein the array comprises at least about 5 wipes.

15. The array of claim 13, wherein each wipe in the array is individually packaged in a pouch, wherein each pouch is attached to an individually packaged absorbent article.

16. An absorbent article combination comprising:
a. a wearable absorbent article;
b. a releasable wrapper wherein the releasable wrapper at least partially surrounds the absorbent article; and
c. a pouch joined to the releasable wrapper, the pouch having a nonwoven wipe disposed therein, wherein the wipe is printed.

17. The absorbent article combination of claim 16, wherein the absorbent article is a sanitary napkin.

18. A package for storing one or more wipes, the package having one or more walls defining an interior storage space and a means for accessing the interior storage space, the package comprising one or more wipes disposed in the interior storage space, wherein at least a portion of the one or more walls being substantially transparent, wherein a portion of at least one of the wipes is disposed adjacent to the substantially transparent wall portion.

19. The package of claim 18, wherein one or more of the wipes is individually packaged in a pouch.

20. The package of claim 18, wherein the package comprises a plurality of wipes.
